(19) United States
(12) Patent Application Publication
(10) Pub. No.: US 2026/0168974 A1
(43) Pub. Date: Jun. 18, 2026

(54) APPARATUS AND METHOD FOR DETECTING OIL FUME

(71) Applicant: LG ELECTRONICS INC., Seoul (KR)

(72) Inventors: Woodeok JOO, Seoul (KR); Moosub KIM, Seoul (KR); Yunguk JANG, Seoul (KR); Narae KIM, Seoul (KR); Wanseop CHOI, Seoul (KR)

(73) Assignee: LG ELECTRONICS INC., Seoul (KR)

(21) Appl. No.: 19/232,082

(22) Filed: Jun. 9, 2025

(30) Foreign Application Priority Data
Dec. 13, 2024 (WO) PCT/KR2024/096879

(51) Int. Cl. G01N 33/00; G01N 15/00; G01N 33/03

(52) U.S. Cl. CPC G01N 33/0031; G01N 33/0063; G01N 33/03; G01N 2015/0026

(57) ABSTRACT

An apparatus for detecting oil fume in the air is disclosed. The apparatus includes: a fine dust sensor configured to detect fine dust; a gas sensor configured to detect gas; and a processor configured to obtain oil fume information using time-series sensor data or non-time-series sensor data obtained from the fine dust sensor and the gas sensor.

This application claims the benefit of International Patent Application No. PCT/KR2024/096879, filed on Dec. 13, 2024, which is hereby incorporated by reference as if fully set forth herein.

### BACKGROUND OF THE DISCLOSURE

### Field of the Disclosure

Embodiments of the present disclosure relate to an apparatus and method for detecting oil fumes, and more particularly to an apparatus and method for detecting the concentration of oil fumes or the presence or absence of oil fumes based on at least two types of sensor data that can be detected in the air.

### Discussion of the Related Art

Oil fumes are among the most common pollutants that may occur in homes and cooking facilities among cooking fumes. The size of such oil fumes falls into the category of ultrafine dust (2.5 μm or less) or fine dust (10 μm or less), and harmfulness to the human body is mitigated through ventilation or air purifier operation. The filter replacement time may be determined by a lifespan estimated based on the user's personal experience. Alternatively, if there is a sensor for detecting a specific harmful material, a method for enabling the sensor to measure the quality of air discharged from an outlet and then generating an alarm based on the result of measurement may be used. However, the lifespan determination method based on the user's personal experience cannot reflect the variability of the filter lifespan depending on the user's environment and behavior. In addition, household vapor filters customized for oil fumes do not have related sensors, so that it is difficult to generate filter replacement alarms based on measurement values obtained from the related sensors.

The present disclosure proposes a sensor capable of detecting oil fumes, a device including the sensor, or a system including the device.

### SUMMARY OF THE DISCLOSURE

Accordingly, the present disclosure is directed to an apparatus and method for detecting oil fumes that substantially obviate one or more problems due to limitations and disadvantages of the related art.

An object of the present disclosure is to provide a sensor for detecting oil fumes, a device including the sensor, or a system including the device, which can determine the filter replacement time for the existing air purifier or can solve the problem encountered in sensing oil fumes included in the air.

Another object of the present disclosure is to provide a sensor that detects oil fumes in the air by designing and training an artificial intelligence (AI) model for sensing the oil fumes, and provides a user notification message, filter lifespan information, or the like based on the sensed result, a device including the sensor, or a system including the device.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as the appended drawings.

To achieve these objects and other advantages and in accordance with the purpose of the invention, as embodied and broadly described herein, an apparatus for detecting oil fume in air may include: a fine dust sensor configured to detect fine dust; a gas sensor configured to detect gas; and a processor configured to obtain oil fume information using time-series sensor data or non-time-series sensor data obtained from the fine dust sensor and the gas sensor.

Additionally or alternatively, the oil fume information may include information about presence or absence of oil fume or information about a concentration of oil fume.

Additionally or alternatively, the processor may be configured to use a training-evaluation model to obtain oil fume information from the time-series sensor data or the non-time-series sensor data.

Additionally or alternatively, the processor may be configured to update the time-series sensor data or the non-time-series sensor data; and update the training-evaluation model using the updated sensor data.

Additionally or alternatively, the processor may output a signal for device operation control according to the oil fume information.

Additionally or alternatively, the processor may output a visual or auditory notification through a human-machine interface according to the oil fume information.

Additionally or alternatively, the processor may output the oil fume information through a human-machine interface; or may transmit the oil fume information to a server or a service application.

Additionally or alternatively, the processor may be configured to transmit the time-series sensor data or the non-time-series sensor data in response to that the oil fume information satisfies a preset condition.

Additionally or alternatively, the processor may be configured to generate lifespan information of an oil fume filter using the oil fume information.

Additionally or alternatively, the processor may be configured to output the generated lifespan information of the oil fume filter through a human-machine interface; or transmit the generated lifespan information of the oil fume filter to a server or a service application.

In accordance with another embodiment of the present disclosure, a method for detecting oil fume in air may include: obtaining time-series or non-time-series fine dust sensor data, wherein the time-series or non-time-series sensor data includes fine dust sensor data and gas sensor data; and obtaining oil fume information using the time-series or non-time-series sensor data.

In accordance with another embodiment of the present disclosure, a non-transitory computer-readable storage medium may include: code configured to execute, by a computer or a processor, the method for detecting oil fume.

The above-described solutions of the present disclosure are only some of the preferred embodiments of the present disclosure, and various embodiments reflecting the technical features of the present disclosure may be derived and understood from the following detailed description of the present disclosure by those skilled in the art.

It is to be understood that both the foregoing general description and the following detailed description of the present disclosure are exemplary and explanatory and are intended to provide further explanation of the disclosure as claimed.

### BRIEF DESCRIPTION OF THE DRAWINGS

The accompanying drawings, which are included to provide a further understanding of the disclosure and are incorporated in and constitute a part of this application, illustrate embodiment(s) of the disclosure and together with the description serve to explain the principle of the disclosure.

FIG. 1 is a diagram illustrating a general indoor ventilation system.

FIG. 2 is a diagram illustrating a time-dependent change in heterogeneous sensing data according to the present disclosure.

FIG. 3 is a flowchart illustrating a structure of a training and evaluation model for oil fume sensing according to the present disclosure.

FIGS. 4 to 9 illustrate operation scenarios of a sensor, device, system, etc. for oil fume sensing according to the present disclosure.

FIG. 10 is a block diagram illustrating a sensing device for sensing oil fume according to the present disclosure.

### DETAILED DESCRIPTION OF THE DISCLOSURE

Description will now be given in detail according to exemplary embodiments disclosed herein, with reference to the accompanying drawings. For the sake of brief description with reference to the drawings, the same or equivalent components may be provided with the same reference numbers, and description thereof will not be repeated. In general, a suffix such as “module” and “unit” may be used to refer to elements or components. Use of such a suffix herein is merely intended to facilitate description of the specification, and the suffix itself is not intended to give any special meaning or function. In the present disclosure, that which is well-known to one of ordinary skill in the relevant art has generally been omitted for the sake of brevity. The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings.

It will be understood that although the terms first, second, etc. may be used herein to describe various elements, these elements should not be limited by these terms. These terms are generally only used to distinguish one element from another.

It will be understood that when an element is referred to as being “connected with” another element, the element can be directly connected with the other element or intervening elements may also be present. In contrast, when an element is referred to as being “directly connected with” another element, there are no intervening elements present.

A singular representation may include a plural representation unless it represents a definitely different meaning from the context.

Terms such as “include” or “has” are used herein and should be understood that they are intended to indicate an existence of several components, functions or steps, disclosed in the specification, and it is also understood that greater or fewer components, functions, or steps may likewise be utilized.

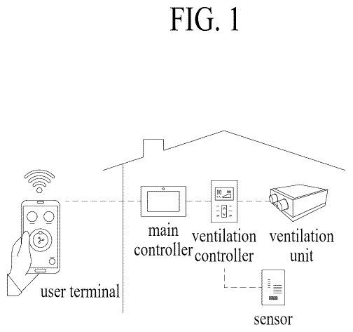

FIG. 1 is a diagram illustrating a general indoor ventilation system.

The indoor ventilation system utilizing sensing data such as fine dust is a smart system that monitors the condition of indoor air in real time and automatically controls the ventilation unit (fan) as needed to purify the air or exchange indoor and outdoor air. This system can be used in various environments such as homes, offices, public spaces, etc.

A main controller called a wall pad is used as a central control device installed in a living space, and controls the ventilation system based on sensor data and user input. Indoor air-quality information can be checked or ventilation settings can be adjusted through the user interface (UI) of the main controller. In addition, the main controller can be linked with a user terminal UE (e.g., a smartphone) to enable remote control and monitoring.

The user terminal UE (or the application installed in the UE) may communicate with the main controller to remotely control the system or check the system status. The user terminal (UE) may deliver information about the indoor air quality to the user in real time through the notification function. For example, if the indoor fine dust concentration exceeds a reference value, the application (app) can send a notification to the user, recommend ventilation, or start automatic execution.

The ventilation controller may directly connect to the main controller (wall pad) or may wirelessly communicate with the main controller and control the ventilation unit (fan). The ventilation controller may control the speed, direction, operation time, etc. of the fan based on sensor data and commands from the main controller

The sensor may measure fine dust (PM2.5, PM10), carbon dioxide (CO₂), volatile organic compounds (VOC), temperature, humidity, etc. Data measured by the sensor may be transmitted to the wall pad and reflected in real-time monitoring and ventilation control. The sensors are installed indoors and outdoors, respectively, so that the status of the outside air can also be measured.

The ventilation unit (fan) is responsible for indoor/outdoor air exchange or indoor air circulation. The ventilation unit may automatically adjust the fan operation status (ON/OFF), speed, etc. according to sensor data and user settings. The ventilation unit may purify outside air using a high-performance filter and then introduce the purified outside air into the room or may discharge indoor polluted air to the outside.

The indoor ventilation system enables automated air quality management. The indoor ventilation system can automatically operate when the indoor air quality deteriorates, thereby maintaining a comfortable environment without user intervention.

In addition, the indoor ventilation system can optimize energy efficiency in terms of minimizing fan operation when the need for ventilation is low, thereby reducing energy consumption. The indoor ventilation system may set a ventilation schedule for each time zone, which can also increase energy efficiency.

In addition, the indoor ventilation system can implement a strategy to improve the indoor air quality by only circulating indoor air when the outside air condition is polluted.

Meanwhile, as another modification of the indoor ventilation system, there is a structure in which the user terminal (UE) is directly connected to the ventilation controller or the sensor. That is, the main controller (wall pad) is not used, and the user terminal (UE) receives sensor data from the sensor and can control the ventilation controller if necessary.

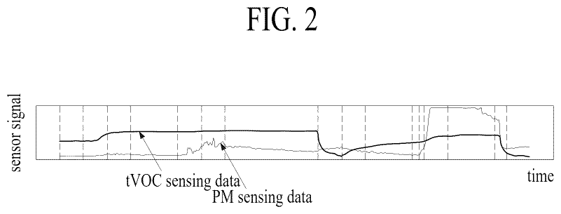

FIG. 2 is a diagram illustrating a time-dependent change in heterogeneous sensing data according to the present disclosure.

A fine dust sensor (PM sensor) (particle sensor) may measure the concentration of particles in the air using a beta-ray method or a light scattering method. Among gas sensors, tVOC (Total Volatile Organic Compounds) sensors reacting to tVOC may include a semiconductor tVOC sensor, an electrochemical tVOC sensor, a contact combustion tVOC sensor, an infrared (IR) absorption/scattering tVOC sensor, etc. Light scattering PM sensors and semiconductor tVOC sensors are mainly applied to home air purifiers. Depending on the tVOC sensor types, tVOC sensors may exhibit different reactivities to target gases, and multi-gas sensors that are arranged in parallel so that a single sensor can detect various e types of gases can also be widely used.

Since the oil fumes are mainly composed of particles of 10 μm or less and exhibits the characteristics of volatile organic compounds, signals can be obtained from not only the PM sensor but also the tVOC sensor. As shown in FIG. 2, PM sensing data and tVOC sensing data may vary over time, and the indoor oil fumes can be sensed using such sensing data.

The PM sensor measures not only oil fumes but also other fine dust or water vapor particles, and since the tVOC sensor also reacts to various organic compounds in addition to oil fumes, the accuracy of oil fume measurement cannot be guaranteed with only the measurement values of individual sensors.

In addition, in addition to the PM sensor and the tVOC sensor, an IAQ sensor, a toluene sensor, an H₂ sensor, an NH₃ sensor, an H₂S sensor, a CO₂ sensor, a NOₓ sensor, a temperature/humidity sensor, etc. can be used as sensors for sensing oil fumes.

In addition, since the method of measuring the presence or concentration of oil fumes using only numerical values of the sensor data can be performed based on rules as to whether the sensor data values are equal to or higher than (or less than) a threshold, the reliability of oil fume measurement may be deteriorated.

The present disclosure aims to provide an oil fume sensing algorithm that senses whether oil fume is generated and the degree of oil fume generation through the artificial intelligence (AI) algorithm based on two types of sensor data. Hereinafter, as a representative example, the oil fume sensing algorithm that senses whether oil fumes are generated and the degree of oil fume generation through the artificial intelligence (AI) algorithm based on PM sensor data and tVOC gas sensor data that are measured at the same time will be described.

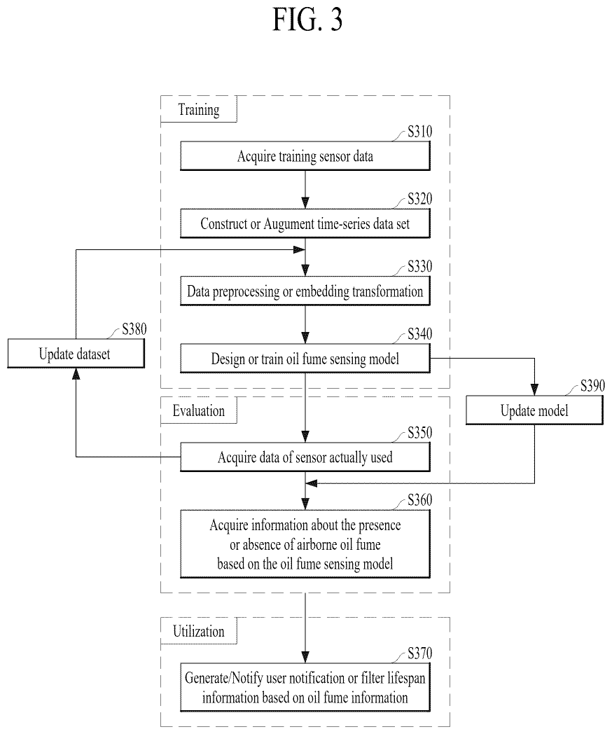

FIG. 3 is a flowchart illustrating a structure of the training and evaluation model for such oil fume sensing according to the present disclosure.

The sensing data acquired by utilizing the oil fume sensing algorithm according to the present disclosure can be utilized for augmenting the training sensor data set, and the improved model may be updated to the device to enable improved oil fume sensing.

In the actual usage environment, the amount of oil fumes obtained through the oil fume sensing model or the hourly vapor change amount acquired through the oil fume sensing model may be immediately transferred to the user, or the user can be notified of the amount of accumulated oil fumes at regular intervals. The user can be notified of the results of the analysis of the user's lifestyle pattern based on the stored accumulated history and total accumulated amount, or filter lifespan information can be generated and the user can be notified thereof.

Hereinafter, the training and evaluation model for oil fume sensing (hereinafter referred to as “oil fume sensing model”) according to the present disclosure will be described.

Referring to FIG. 3, in order, a method for using the oil fume sensing model may include: designing and training the oil fume sensing model; measuring the air using the result of such training; acquiring oil fume information about the presence or absence of oil fumes or the concentration of oil fume; and performing either evaluation of generating a user notification based on the acquired oil fume information or evaluation of acquiring the filter lifespan information.

Hereinafter, the oil fume sensing model will be described as performing the procedure illustrated in FIG. 3 for convenience of description, but in the case where the oil fume sensing model is implemented as a device configuration such as a sensor or a sensing device, the sensor, the sensing device or a processor thereof may be configured to perform the following procedures. In addition, the oil fume sensing model may be executed or driven on, for example, a cloud server rather than a sensor or a sensing device.

The oil fume sensing model may obtain sensor data for training (S310). For example, the sensor data for training may include fine dust (PM) sensing data obtained through the PM sensor and tVOC sensing data obtained through the tVOC sensor.

The oil fume sensing model can construct or augment a time-series data set over time using the training sensor data (S320).

The time-series data set may be constructed using at least some of the training sensor data, that is, the sensing data acquired through each sensor.

In addition, it may be difficult to construct a sufficient amount of training sensor data with sensor data acquired in a limited environment. Accordingly, the oil fume sensing model can augment the acquired sensor data to secure the robustness of the oil fume sensing model. As data augmentation techniques, various techniques such as amplitude scaling, time scaling, noise addition, distortion, cutting, and movement can be used. In addition, techniques based on generative artificial intelligence (AI) such as GAN and VAE can be used as data augmentation techniques.

Thereafter, the oil fume sensing model may preprocess the time-series data set of the sensor data or may perform embedding transformation of the time-series data set of the sensor data.

In data preprocessing, PM sensor data can be converted into an appropriate level of signals through amplitude scaling or amplitude shifting. In addition, tVOC sensor data can be used to convert a signal range through amplitude adjustment or equalization of raw data. The scaling factor may be a preset constant or a maximum value, average value, etc. during a specific period.

Embedding transformation is a process of extracting new features from the existing feature relationships or changing the extracted features into main features. In the embedding transformation process, features such as ratios between specific features, Nth-order differential values of individual features, and Fourier transform line segments can be used. In addition, in the embedding transformation process, features effectively reduced or converted through dimension reduction such as PCA, UMAP, and VAE can be defined and used as input layers.

The oil fume sensing model can be designed or acquired using data acquired through S310 to S330, and trained.

The training model for training the oil fume sensing model can be selected by considering the problem definition, the characteristics of a dataset, the form of the final output, the structure and performance, and the environmental conditions including the model sizes. During supervised training, a non-time series model, a time-series model, or a non-time series-time series composite model can be used, and the unsupervised training or the self-supervised training can be used.

As a model that uses sensing data at individual points in time as features in a non-time series manner, a deep neural network (DNN) model such as multilayer perception (MLP), a random forest or a support vector machine (SVM) can be used as representative models.

When utilizing the time-series characteristics in the dataset, the change trend of each sensing data over time is bundled as input data or embedding, so that a time-series model (e.g., RNN, LSTM, time-series transformer, etc.) can be trained. Unlike a non-time series model that uses data at a single point in time, training can be performed sequentially according to the time flow of the data, or the input data can be reconstructed into data that includes time information and the resultant data can be used for training. Such training can be performed through convolutional neural network (CNN) or long-short term memory (LSTM) models, etc.

In the case of a binary classification problem that determines only the presence or absence of oil fumes, the dimension of the final output vector is 2, and in the case of multi-classification, the dimension is adjusted according to the number of classifications.

In the case of a regression model such as the prediction of the concentration of oil fumes, the model may be configured to derive continuous output values by excluding the softmax layer of the final stage.

A representative example of unsupervised training or self-supervised training may be a clustering method. When label information is absent or provided in a limited manner, a model can be trained to perform tasks such as classification by autonomously understanding the internal characteristics of data.

By training various models, parameters for each model can be determined and the determined parameters can be used for evaluation.

In terms of evaluation, when the design or training of the oil fume sensing model is completed, the oil fume sensing model can perform an evaluation task to obtain oil fume information such as the presence or absence of oil fumes in the air or the concentration of oil fumes.

The oil fume sensing model can obtain sensor data from sensors (S350).

The oil fume sensing model can obtain oil fume information such as the presence or absence of oil fumes in the air or the concentration of oil fumes using the oil fume sensing model trained based on the obtained sensor data (S360).

Thereafter, the oil fume sensing model may generate user notifications or filter lifespan information based on the oil fume information (S370).

The user notification may include a notification that airborne oil fumes are detected, a notification about the airborne oil fume concentration, etc. The filter lifespan information may be generated using the oil fume concentration over time and the information of the ventilation fan (e.g., rotation speed, air permeability according to such rotation, etc.). If the ventilation unit (e.g., an air purifier) includes a specialized oil fume filter, the obtained oil fumes-based filter lifespan information may accurately alert the user to filter replacement time.

In addition, the oil fume sensing model may update the dataset (S380) or may perform a model update (S390) by training the model using the updated dataset. As a result, the accuracy or reliability of obtaining airborne oil fume information, including information about the presence or absence of oil fumes or information about the concentration of oil fumes, may increase.

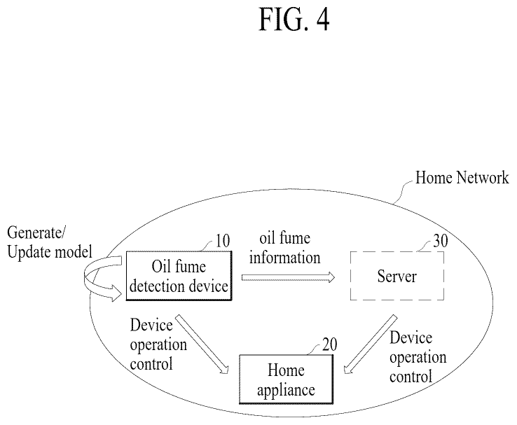

FIGS. 4 to 9 illustrate operation scenarios of the sensor, device, system, etc. for oil fume sensing according to the present disclosure.

FIG. 4 illustrates a system for oil fume sensing according to the present disclosure. FIG. 4 illustrates a first scenario of on-device-only operation in which the oil fume detection device 10 directly performs the operation or procedure of FIG. 3 described above.

The oil fume detection device 10 may be configured to design, generate, develop, or train a vapor sensing model (i.e., a training-evaluation model for oil fume sensing) described with reference to FIG. 3, and may process sensor data acquired through the oil fume sensing model to acquire oil fume information.

The oil fume detection device 10 may store sensor data and may record the sensor data in a time-series manner. Time-series sensor data can be used to obtain oil fume information. In addition, non-time-series sensor data can be used to obtain oil fume information.

The oil fume detection device 10 may be configured as a single sensor module or as a sensor station including a plurality of sensors.

In addition, the oil fume detection device 10 may be configured to update the oil fume sensing model using the obtained sensor data.

In addition, the oil fume detection device 10 may be configured to transmit device operation control information to a home appliance 20 including the ventilation unit. For such device operation control, whether to transmit information based on the obtained oil fume information may be determined or information to be included in the oil fume information may be determined.

For example, the device operation control information may be transmitted to the home appliance 20 when oil fume information exceeding a predetermined range of difference from the oil fume information formed based on the previous device operation control is obtained. Alternatively, the device operation control information may be configured to be periodically transmitted to the home appliance 20 regardless of content of the oil fume information.

For example, the device operation control information may include information on the degree of necessity for ventilation or purification, for example, the necessity of ventilation or air purification at the first level, the necessity of ventilation or air purification at the second level, etc. For example, the device operation control information may include oil fume information. The vapor information may include information on whether oil fumes exist in the air or information about the concentration of oil fumes. When the device operation control information includes oil fume information, the home appliance 20 may directly control the device operation according to the oil fume information.

Additionally, the home network may include a server 30. The server 30 may include a service application (app) for indoor air management. In addition, the server 30 may not be a physical configuration. That is, the server 30 may include a remote server connected to the home network.

There is no limitation on the management entity of the server 30, and the server 30 may not be managed by the user of the oil fume detection device 10.

The oil fume detection device 10 may be configured to transmit the acquired oil fume information to the server 30. Transmission of the vapor information may be performed only when a preset condition is satisfied, or may be performed periodically. The server 30 may store the oil fume information and may record the stored information in a time-series manner.

The server 30 may be configured to transmit device operation control information to the home appliance 20 based on the received oil fume information. The device operation control information may include information required for determining whether to transmit necessary information based on the obtained oil fume information, or may be used to determine information to be included in the oil fume information. For example, the device operation control information may include information on the degree of necessity of ventilation or purification, such as the need for ventilation or air purification at the first level, the need for ventilation or air purification at the second level, etc. For example, the device operation control information may include oil fume information. The oil fume information may include information on whether oil fumes are present in the air or the concentration of oil fumes. When the device operation control information includes oil fume information, the home appliance 20 may directly control the device operation according to the oil fume information.

The home appliance 20 may be configured to control a motor or a fan according to the device operation control information received from the oil fume detection device 10 or the server 30. Home appliances 20 include devices configured to discharge indoor air to the outside or configured to filter air, such as ventilators, air purifiers, air-conditioners, or heat exchangers, but the types thereof are not limited thereto.

The oil fume detection device 10 may store oil fume information and may record the oil fume information in a time-series manner. The oil fume detection device 10 may be configured to predict the remaining lifespan of a filter (e.g., an oil-vapor-specified filter) using the time-series vapor information. In predicting the remaining lifespan of the filter, information about the fan speed of the home appliance 20 or the fan speed information according to time, the amount of air passing through the filter or the amount of air passing through the filter according to time, information about the filter lifespan, or information about the amount of air passing through the filter (or the amount of oil fume passing through the filter) can be used.

Alternatively, the server 30 may store oil fume information, and may record the oil fume information in a time-series manner. The server 30 may be configured to predict the remaining lifespan of a filter (e.g., an oil-vapor-specified filter) using the time-series vapor information. In predicting the remaining lifespan of the filter, information on the fan speed of the home appliance 20 or the fan speed over time, the amount of air passing through the filter or the amount of air passing through the filter over time, the filter lifespan, or the amount of air passing through the filter (or the amount of oil fumes passing through the filter), etc. can be used.

The prediction of the total lifespan of the filter may be as follows.
1. The filter lifespan can be estimated by comparing the total amount of oil fumes generated from the predefined filter with the accumulated value of oil fumes generated during actual use.
2. The filter lifespan can be estimated by comparing the total available time of oil fumes generated from the predefined filter with the accumulated value of the number or time of oil fume generation events.

At this time, the filter specifications such as the total capacity of oil fumes or total available time of oil fume can be defined by considering the oil fume removal efficiency, oil fume capture capacity, pressure loss, etc.

Meanwhile, in the system illustrated in FIG. 4, the oil fume detection device 10 obtains oil fume information, but may not transmit device operation control information to the home appliance 20. In addition, the oil fume detection device 10 may transmit the oil fume information to the server 30. That is, the oil fume detection device 10 may perform only the role of sensing or monitoring oil fumes present in indoor air.

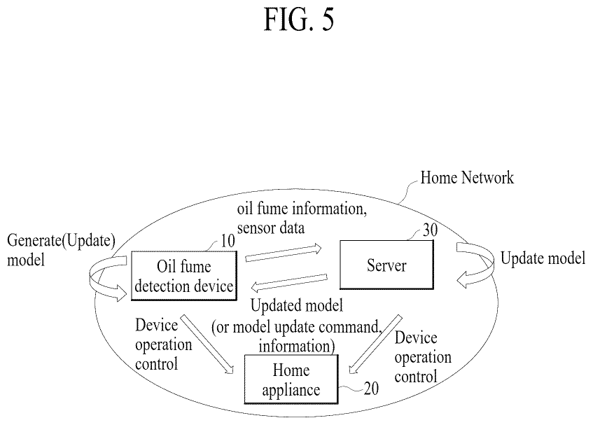

FIG. 5 illustrates a system for oil fume sensing according to the present disclosure. FIG. 5 illustrates a first scenario of the on-device-server collaboration in which the oil fume detection device 10 and the server 30 perform the operations or procedures of FIG. 3 described above.

FIG. 5 illustrates a scenario in which the oil fume detection device 10 designs or generates the oil fume sensing model, and the update of the oil fume sensing model is performed by the server 30. In addition, the oil fume detection device 10 is configured to transmit oil fume information or sensor data to the server 30, and the sensor data can be used to update the oil fume sensing model.

The server 30 may include a service application (app) for indoor air management. In addition, the server 30 may not be a physical configuration. That is, the server 30 may include a remote server connected to the home network.

The server 30 may be configured to generate update information for updating the oil fume sensing model based on the oil fume information or sensor data (i.e., “dataset” of FIG. 3) received from the oil fume detection device 10.

When sensor data is received from the oil fume detection device 10, the server 30 may be configured to obtain oil fume information using the received sensor data.

The server 30 may store sensor data received from the oil fume detection device 10, and may record the sensor data in a time-series manner. Time-series sensor data may be used to obtain the oil fume information. In addition, non-time-series sensor data may be used to obtain the oil fume information.

Here, the update information may include information that can obtain an updated oil fume sensing model based on the vapor sensing model pre-stored in the oil fume detection device 10. Accordingly, the oil fume detection device 10 may be configured to update the oil fume sensing model using the received update information.

Meanwhile, the server 30 may be configured to receive the initial oil fume sensing model from the oil fume detection device 10. Accordingly, the server 30 may store the same vapor sensing model as the oil fume detection device 10. Thus, the server 30 may be configured to directly update the oil fume sensing models using the update information.

The device operation control information or other content that are not described with reference to FIG. 5 may be applied in the same or similar manner as the control and operation of the scenario described with reference to FIG. 4, and as such redundant description thereof will herein be omitted from FIG. 5 for the sake of simplicity.

Meanwhile, in the system illustrated in FIG. 5, the oil fume detection device 10 may obtain oil fume information and may transmit the obtained information to the server 30, but may not transmit device operation control information to the home appliance 20. In addition, the server 30 may not transmit device operation control to the home appliance 20. In other words, the oil fume detection device 10 may only perform the role of sensing or monitoring vapor present in indoor air.

Meanwhile, in the system illustrated in FIG. 5, the oil fume detection device 10 may acquire the oil fume information and transmit the oil fume information to the server 30, but may not transmit the device driving control to the home appliance 20. In addition, the server 30 may also not transmit the device driving control to the home appliance 20. That is, the oil fume detection device 10 may perform only the role of sensing or monitoring the oil fumes present in the indoor air.

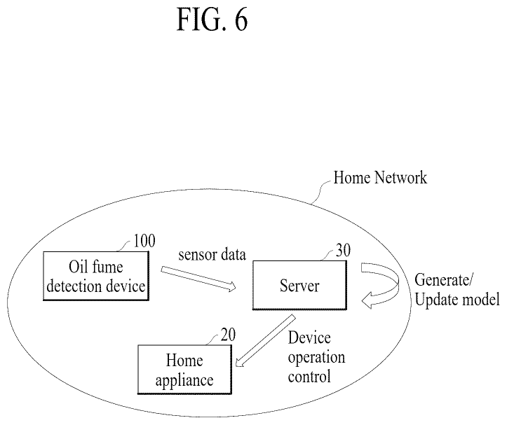

FIG. 6 illustrates a system for oil fume sensing according to the present disclosure. FIG. 6 illustrates a first scenario of the server operation in which the server 30 mainly performs the operations or procedures of FIG. 3 described above.

Unlike FIGS. 4 and 5, FIG. 6 illustrates a scenario in which the oil fume detection sensor 100 instead of the oil fume sensing device is included in the home network.

In the scenario of FIG. 6, the oil fume detection sensor 100 may be configured to acquire sensor data and transmit the acquired sensor data to the server 30. The oil fume detection sensor 100 may include a fine dust sensor and a tVOC sensor. Accordingly, the sensor data may include fine dust sensor data and tVOC sensor data. That is, sensor data as shown in FIG. 2 may be acquired and transmitted.

In addition, unlike FIGS. 4 and 5, the server 30 may be configured to directly design, generate, or update the oil fume sensing model. That is, the server 30 of FIG. 6 is configured to operate almost similarly to the oil fume sensing device 10 of FIG. 4.

The scenario of FIG. 6 has advantages in that it is not necessary to separately provide the oil fume sensing device, and thus, it is possible to easily and inexpensively implement oil fume sensing and control functions in the home network as well as to easily control the home appliances using the sensed results. That is, since the server 30 is configured as a service application (app), and the oil fume sensing model is designed, created, or updated in a remote server, data processing load or the required performance for the data processing load is not high in a local stage (or local area). However, since information from the oil fume detection sensor 100 needs to be continuously transmitted to the server 30, the network quality within the home network needs to be maintained excellent and uniform.

The device operation control information or other content not described with reference to FIG. 6 may be applied in the same or similar manner as the control and operation of the scenario described with reference to FIG. 4, and as such redundant description thereof will herein be omitted from FIG. 6 for the sake of simplicity.

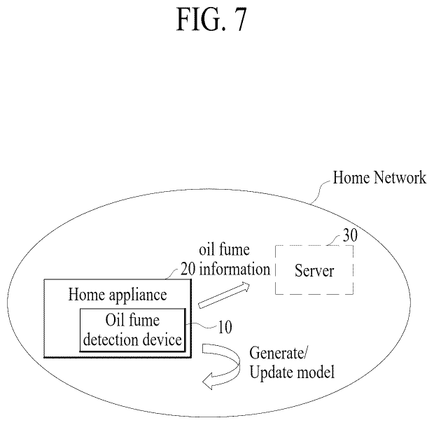

FIG. 7 illustrates a system for oil fume sensing according to the present disclosure. FIG. 7 illustrates a second scenario of on-device-only operation in which the oil fume detection device 10 directly performs the operation or procedure of FIG. 3, but is embedded in the home appliance 20.

The oil fume detection device 10 is embedded in the home appliance 20. Accordingly, the oil fume detection device 10 operates almost similarly to the oil fume detection device 10 of FIG. 4, and although not illustrated, the oil fume detection device 10 may be configured to perform device operation control of the home appliance 20 according to oil fume information.

In addition, the home network may include the server 30. The server 30 may include a service application (app) for indoor air management. In addition, the server 30 may not be a physical configuration. That is, the server 30 may include a remote server connected to the home network.

The oil fume detection device 10 or the home appliance 20 may be configured to transmit oil fume information to the server 30. The server 30 may store the oil fume information and record such information in a time-series manner.

The server 30 may be configured to transmit device operation control information to the home appliance 20 based on the received oil fume information.

The device operation control information or other contents that are not described with reference to FIG. 7 may be applied in the same or similar manner as the control and operation of the scenario described with reference to FIG. 4, and as such redundant description thereof will herein be omitted from FIG. 7 for the sake of simplicity.

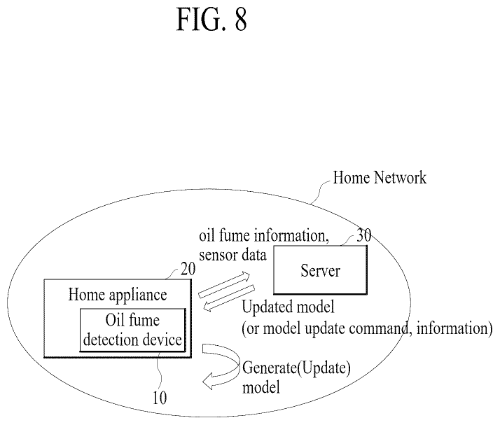

FIG. 8 illustrates a system for oil fume sensing according to the present disclosure. FIG. 8 illustrates a second scenario of on-device-server collaboration operation in which both the oil fume detection device 10 embedded in the home appliance 20 and the server 30 implement the operation or procedure of FIG. 3 described above.

The oil fume detection device 10 is embedded in the home appliance 20. The oil fume detection device 10 of FIG. 8 designs or generates the oil fume sensing model, and the update of the oil fume sensing model is performed by the server 30. Accordingly, the oil fume detection device 10 can operate almost similarly to the oil fume detection device 10 of FIG. 5.

The server 30 may include a service application (app) for indoor air management. In addition, the server 30 may not be a physical configuration. That is, the server 30 may include a remote server connected to the home network.

The server 30 may store the same vapor sensing model as the oil fume detection device 10, and accordingly, the server 30 may be configured to directly update the oil fume sensing models using the update information.

The device operation control information or other contents that are not described with reference to FIG. 8 may be applied in the same or similar manner as the control and operation of the scenario described with reference to FIG. 54, and as such redundant description thereof will herein be omitted from FIG. 8 for the sake of simplicity.

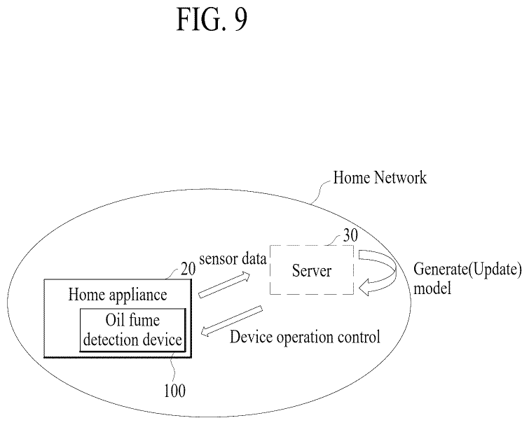

FIG. 9 illustrates a system for sensing vapor according to the present disclosure. FIG. 9 illustrates a second scenario of server operation in which the server 30 mainly performs the operation or procedure of FIG. 3 described above.

The oil fume detection sensor 100 is embedded in the home appliance 20. The oil fume detection sensor 100 may be configured to acquire sensor data and transmit the acquired sensor data to the server 30. The oil fume detection sensor 100 may include the fine dust sensor and the tVOC sensor. Therefore, the sensor data may include fine dust sensor data and tVOC sensor data. That is, sensor data such as FIG. 2 may be acquired and transmitted.

In addition, unlike FIGS. 4 and 5, the server 30 may be configured to directly design, generate, or update the oil fume sensing model. That is, the server 30 of FIG. 9 is configured to operate almost similarly to the oil fume sensing device 10 of FIG. 4.

The device operation control information or other contents that are not described with reference to FIG. 9 may be applied in the same or similar manner as the control and operation of the scenario described with reference to FIG. 4 or FIG. 6, and as such redundant description thereof will herein be omitted from FIG. 9 for the sake of simplicity.

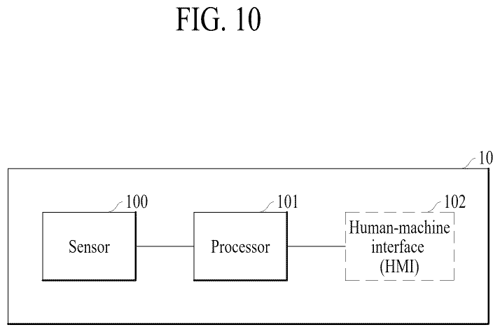

FIG. 10 illustrates a block diagram of a sensing device for oil fume sensing according to the present disclosure.

Referring to FIG. 10, the sensing device 10 may include a sensor 100 and a processor 101.

The sensing device 10 is a device for detecting oil fumes in the air.

The sensor 100 may be configured to acquire at least two types of sensor data and may acquire air characteristics in the air. Preferably, the sensor 100 may include a fine dust sensor configured to sense fine dust and a gas sensor configured to sense gas. The gas sensor may include the tVOC sensor.

The processor 101 may be configured to acquire oil fume information using time-series sensor data or non-time-series sensor data acquired from the fine dust sensor and the gas sensor. Here, the oil fume information may include the presence or absence of oil fumes or the concentration of oil fumes.

The processor 101 may be configured to use the training-evaluation model to acquire oil fume information from the time-series sensor data or the non-time-series sensor data. Here, the training-evaluation model is a model described with reference to FIG. 3 and corresponds to an “oil fume sensing model.”

The processor 101 may be configured to update the time-series sensor data or the non-time-series sensor data, and update the training-evaluation model using the updated sensor data.

Meanwhile, the update of the training-evaluation model may be triggered by the server 30 illustrated in FIGS. 4 to 9, rather than the sensing device 10. According to this embodiment, the processor 101 may be configured to receive information about the updated training-evaluation model from the server 30 according to the time-series sensor data or the non-time-series sensor data. The information about the updated training-evaluation model may include information that can obtain the updated training-evaluation model based on the prestored training-evaluation model. Accordingly, the processor 101 may be configured to update the training-evaluation model using the information about the updated training-model.

The processor 101 may be configured to output a signal for device operation control according to the acquired vapor information. The signal for device operation control may include a control signal for driving or rotating the fan of the device (e.g., the air purifier or the ventilation unit). Accordingly, the air purifier or the ventilation unit may drive the fan, and may filter out oil fumes by allowing indoor air to pass through the filter, so that the air purifier or the ventilation unit can filter out oil fumes from indoor air by discharging indoor air to the outside.

The processor 101 may be configured to output a visual or auditory notification through a human-machine interface (HMI) according to the acquired vapor information. The human-machine interface (HMI) may output not only a visual or auditory notification, but also a haptic notification or various other notifications.

The processor 101 may be configured to output the acquired oil fume information through an audiovisual human-machine interface, or to transmit the oil fume information to the server or the service application (app).

The processor 101 may be configured to output the acquired oil fume information through a human-machine interface (HMI), or to transmit the oil fume information to the server 30 or the service application (app). Thereby, the oil fume information may be transmitted to a user or a manager who manages an indoor ventilation system including the sensor device 10.

The processor 101 may be configured to transmit the time-series sensor data or the non-time-series sensor data when the acquired oil fume information satisfies a preset condition. The time-series sensor data or the non-time-series sensor data may be transmitted to the server 30 or the service application (app).

In addition, the processor 101 may be configured to generate lifespan information of the oil fume filter using the acquired oil fume information. The processor 101 may be configured to output the lifespan information of the oil fume filter through the human-machine interface (HMI), or to transmit the lifespan information of the oil fume filter to the server 30 or the service application (app).

In addition, the sensing device 10 may additionally include the human-machine interface (HMI) 102. The human-machine interface (HMI) 102 includes a means for providing a visual or auditory notification to a user, and may include, for example, a display, a speaker (buzzer), or an LED light. The notification obtained from the HMI 102 may include not only a visual or auditory notification, but also a haptic notification such as vibration, without being limited thereto.

For example, if it is determined that the oil fume concentration exceeds a threshold, the processor 101 can control the HMI 102 to provide a visual notification, an auditory notification, or a haptic notification.

Although not described with reference to FIG. 10, the sensing device 10 can perform the operations according to the embodiments shown in FIGS. 2 to 9 described above.

In addition, as another aspect of the present disclosure, the operation of the proposed technology described above may be provided as code that may be implemented, realized, or executed by a “computer” (a generic concept including a system on chip (SoC) or a (micro) processor) or a computer-readable storage medium, a computer program product, or the like storing or containing the code. The scope of the present disclosure is extendable to the code or the computer-readable storage medium or the computer program product storing or containing the code.

Detailed descriptions of preferred embodiments of the present disclosure disclosed as described above have been provided such that those skilled in the art may implement and realize the present disclosure. Although the present disclosure has been described above with reference to preferred embodiments, those skilled in the art will understand that various modifications and changes can be made to the present disclosure set forth in the claims below. Accordingly, the present disclosure is not intended to be limited to the embodiments shown herein but is to be accorded the widest scope consistent with the principles and novel features disclosed herein.

As is apparent from the above description, the present disclosure has the following technical effects.

According to the present disclosure, information on oil fumes in the air (i.e., information about detection of the presence or absence of oil fumes in the air or information about oil fume concentration) can be measured.

The embodiments of the present disclosure can accurately measure or estimate information about oil fumes in the air.

The embodiments of the present disclosure can update the oil fume sensing model using information about oil fumes measured or estimated in the air.

In addition, the embodiments of the present disclosure can control the device for ventilation or air purification using information about measured or estimated oil fume in the air.

It will be apparent to those skilled in the art that various modifications and variations can be made in the present disclosure without departing from the spirit or scope of the disclosure. Thus, it is intended that the present disclosure covers the modifications and variations of this disclosure provided they come within the scope of the appended claims and their equivalents.

1. An apparatus for detecting oil fume in air comprising:
a fine dust sensor configured to detect fine dust;
a gas sensor configured to detect gas; and
a processor configured to obtain oil fume information using time-series sensor data or non-time-series sensor data obtained from the fine dust sensor and the gas sensor.

2. The apparatus according to claim 1, wherein:
the oil fume information includes information about presence or absence of oil fume or information about a concentration of oil fume.

3. The apparatus according to claim 1, wherein the processor is configured to:
use a training-evaluation model to obtain oil fume information from the time-series sensor data or the non-time-series sensor data.

4. The apparatus according to claim 3, wherein the processor is configured to:
update the time-series sensor data or the non-time-series sensor data; and
update the training-evaluation model using the updated sensor data.

5. The apparatus according to claim 1, wherein the processor is configured to:
output a signal for device operation control according to the oil fume information.

6. The apparatus according to claim 1, wherein the processor is configured to:
output a visual or auditory notification through a human-machine interface according to the oil fume information.

7. The apparatus according to claim 1, wherein the processor is configured to:
output the oil fume information through a human-machine interface; or
transmit the oil fume information to a server or a service application.

8. The apparatus according to claim 1, wherein the processor is configured to:
transmit the time-series sensor data or the non-time-series sensor data in response to that the oil fume information satisfies a preset condition.

9. The apparatus according to claim 1, wherein the processor is configured to:
generate lifespan information of an oil fume filter using the oil fume information.

10. The apparatus according to claim 9, wherein the processor is configured to:
output the generated lifespan information of the oil fume filter through a human-machine interface; or
transmit the generated lifespan information of the oil fume filter to a server or a service application.

11. A method for detecting oil fume in air comprising:
obtaining time-series or non-time-series fine dust sensor data, wherein the time-series or non-time-series sensor data includes fine dust sensor data and gas sensor data; and
obtaining oil fume information using the time-series or non-time-series sensor data.

12. The method according to claim 11, wherein the oil fume information includes:
information about presence or absence of oil fume or information about a concentration of oil fume.

13. The method according to claim 11, further comprising:
using a training-evaluation model to obtain oil fume information from the time-series sensor data or the non-time-series sensor data.

14. The method according to claim 13, further comprising:
updating the time-series sensor data or the non-time-series sensor data; and
updating the training-evaluation model using the updated sensor data.

15. The method according to claim 11, further comprising:
outputting a signal for device operation control according to the oil fume information.

16. The method according to claim 11, further comprising:
outputting a visual or auditory notification through a human-machine interface according to the oil fume information.

17. The method according to claim 11, further comprising:
outputting the oil fume information through a human-machine interface; or
transmitting the oil fume information to a server or a service application.

18. The method according to claim 11, further comprising:
transmitting the time-series sensor data or the non-time-series sensor data in response to that the oil fume information satisfies a preset condition.

19. The method according to claim 11, further comprising:
generating lifespan information of an oil fume filter using the oil fume information.

20. A non-transitory computer-readable storage medium comprising:
code configured to execute, by a computer or a processor, the method according to claim 11.
